(19) United States
(12) Patent Application Publication
(10) Pub. No.: US 2019/0328829 A1
(43) Pub. Date: Oct. 31, 2019

(54) TRIPARTITE COMBINATION THERAPY FOR PROSTATE CANCER

(71) Applicants: UNIVERSITY OF MARYLAND, BALTIMORE, Baltimore, MD (US); HUMANETICS CORPORATION, Edina, MN (US)

(72) Inventors: Zeljko Vujaskovic, Baltimore, MD (US); Michael Kaytor, Maplewood, MN (US); Isabel Lauren Jackson, Baltimore, MD (US); Javed Mahmood, Ellicott City, MD (US)

(21) Appl. No.: 16/311,056

(22) PCT Filed: Jun. 28, 2017
(86) PCT No.: PCT/US2017/039693

§ 371 Date: Dec. 18, 2018

Related U.S. Application Data
(60) Provisional application No. 62/356,806, filed on Jun. 30, 2016.

(51) Int. Cl. A61K 38/09; A61K 45/06; A61K 31/353

(52) U.S. Cl. CPC A61K 38/09; A61K 31/353; A61K 45/06

(57) ABSTRACT

A treatment regimen for a patient diagnosed with intermediate to high risk prostate cancer. The regimen is a combination therapy that includes (-) fractionated radiation therapy, (-) administration of a therapeutic amount of genistein, and (-) administration of a therapeutic amount of a gonadotropin-releasing hormonal oncologic. The treatment regimen allows the gonadotropin-releasing hormonal oncologic to be administered at a reduced dosing regimen relative to the conventional androgen deprivation therapy dosing regimen for the oncologic with minimal reduction in efficacy, thereby allowing adjustments to the androgen deprivation therapy component of the treatment regimen as necessary and appropriate for alleviating the adverse side effects that accompany androgen deprivation therapy.

### GOVERNMENT SUPPORT

This invention was made with government support under STTR Grant Number R41CA186431 awarded by the US Department of Health and Human Services, National Cancer Institute. The government has certain rights in this invention.

### FIELD OF INVENTION

The invention relates to methods of improving the effectiveness of treatment for prostate cancer.

### BACKGROUND

Prostate cancer is among the most common cancers in men in the United States, with more than 200,000 men in the United States diagnosed annually with prostate cancer, approximately 10% of whom die from the disease. Standard treatments include surgery, chemotherapy, and combination hormone and radiation therapy (RT). Localized prostate carcinoma is sensitive to conventional radiotherapy, but residual disease often causes clinical relapse when used as the sole treatment.

The current standard of care for treatment of prostate carcinoma patients at high risk for relapse or metastatic prostate cancer is a combination of radiation therapy (RT) and androgen deprivation therapy (ADT), typically either by luteinizing hormone releasing hormone (LHRH) agonists or by bilateral orchidectomy. This combination therapy is also widely used and accepted for treatment of intermediate risk prostate carcinoma patients.

Androgen deprivation therapy, while highly beneficial in the treatment of prostate cancer, is accompanied by substantial adverse side effects, including the common side effects of dizziness, headache, sweating and hot flashes, depression and moodiness, change in weight, general pain including sore breasts or testicles, restlessness, difficulty urinating, itching and peeling of the skin, breast development, abnormal sensations in fingers and toes, fatigue, weakness, lack of muscle strength, changes in sexual desire, and erectile dysfunction, and the more serious side effects of severe depression, suicidal thoughts, seizures, diabetes, urinary tract blockage, stroke, sudden death, heart rhythm changes, liver damage, and decrease in bone mass potentially leading to osteoporosis.

Accordingly, a substantial need exists for an improved treatment regimen for prostate carcinoma, preferably one that eliminates or alleviates the adverse side effects that accompany androgen deprivation therapy.

### SUMMARY OF THE INVENTION

The invention is directed to a tripartite treatment regimen for a patient diagnosed with prostate cancer. The regimen is a combination therapy that includes (-) fractionated radiation therapy, (-) administration of a therapeutic amount of genistein, and (-) administration of a therapeutic amount of a gonadotropin-releasing hormonal oncologic.

The regimen preferably involves administration of the gonadotropin-releasing hormonal oncologic at a reduced dosing regimen relative to conventional dosing regimens for androgen deprivation therapy. The reduction in dosing regimen can including a reduction in unit dose, frequency of administration, duration of administration, and combinations thereof.

### DETAILED DESCRIPTION OF A PREFERRED EMBODIMENT

### Definitions

As utilized herein, including the claims, the phrase “Combination Therapy” means administration of two or more different medical treatments in overlapping regimens so that the subject is simultaneously exposed to both treatments. By way of example, the different medical treatments may be two different pharmaceutical agents, a pharmaceutical agent and radiation therapy, a pharmaceutical agent and a surgical procedure, different pharmaceutical agents and radiation therapy, etc.

As utilized herein, including the claims, the phrase “Conventional Androgen Deprivation Therapy Dosing Regimen” means the dosing regimen generally accepted by the medical community and governmental regulatory agencies as safe and effective for use in androgen deprivation therapy for treatment of prostate cancer.

As utilized herein, including the claims, the phrase “Dosing Regimen” means a set of unit doses (typically more than one) that are administered individually separated by periods of time. The recommended set of doses (i.e., amounts, timing, duration, route of administration, etc.) for a particular pharmaceutical agent or radiation therapy constitutes its dosing regimen.

As utilized herein, including the claims, the phrase “Therapeutically Effective Amount” of a pharmaceutical agent or combination of agents means an amount of agent(s) which confers a therapeutic effect on the treated subject, at a reasonable benefit/risk ratio applicable to any medical treatment. The specific therapeutically effective amount for any particular subject may depend upon a variety of factors including the disorder being treated, the severity of the disorder, the age, body weight, general health, sex and diet of the subject; the time of administration, the route of administration, and like factors as are well known in the medical arts.

As utilized herein, including the claims, the term “Treatment” means any medical treatment, including medical procedures, radiation therapy, administration of a pharmaceutical agent, etc., that partially or completely alleviates, ameliorates, relieves, inhibits, delays onset of, reduces severity of and/or reduces incidence of one or more symptoms or features of a particular disease, disorder, and/or condition.

As utilized herein, including the claims, the terms “Unit Dose” or “Dose” means a discrete administration of radiation or a pharmaceutical agent, typically in the context of a dosing regimen.

### Description

The invention is a tripartite combination treatment regimen for a patient diagnosed with prostate cancer. The regimen includes (-) fractionated radiation therapy, (-) administration of a therapeutic amount of genistein, and (-) administration of a therapeutic amount of a gonadotropin-releasing hormonal oncologic.

Active Agents

Testosterone is a known stimulus for cancerous cells of the prostate. Suppressing testosterone secretion or inhibiting the actions of testosterone is an effective component of prostate cancer therapy. A number of leutinizing hormone-releasing hormone (LHRH) agonists suitable for use in androgen deprivation therapy for prostate cancer and suitable for use in this invention are commercially available, including buserelin, goserelin, leuprolide and triptorelin. Preferred gonadotropin-releasing hormonal oncologics for use in this tripartite therapy are the gonadotropin-releasing hormone receptor agonists such as leuprolide.

Leuprolide acetate, sold in the United States by Abbvie Inc. of North Chicago, Ill. under the trademark Lupron® and Lupron® depot, is a synthetic nonapeptide analog of naturally occurring gonadotropin-releasing hormone (GnRH or LHRH). Leuprolide acetate is an LHRH superagonist that eventually suppresses LH secretion by the pituitary. Leuprolide acetate acts as a potent inhibitor of gonadotropin secretion, resulting in suppression of ovarian and testicular steroidogenesis. In humans, administration of leuprolide acetate results in an initial increase in circulating levels of luteinizing hormone (LH) and follicle stimulating hormone (FSH), leading to a transient increase in levels of the gonadal steroids (testosterone and dihydrotestosterone in males, and estrone and estradiol in premenopausal females). Continuous administration of leuprolide acetate results in decreased levels of LH and FSH. In males, testosterone is reduced to castrate levels (below 50 ng/dL).

Other commercially available LHRH agonists sold for use in the treatment of prostate cancer are Eligard®, sold in the United States by Sanofi of Paris, France, and triptorelin available from Watson Laboratories, Inc. of Corona, Calif. under the trademark Trelstar®.

Genistein belongs to the pharmacological classes of soy isoflavone, flavonoid, polyphenol and phytoestrogen. It is also known as 5,7-dihydroxy-3-(4-hydroxyphenyl)-chromen-4-one (IUPAC), 5,7-dihydroxy-3-(4-hydroxyphenyl)-4H-1-benzopyran-4-one, 5,7,4′-trihydroxyisoflavone, 4′,5,7-trihydroxyisoflavone, Genestein, Prunetol, Sophoricol and Differenol A. It has a Molecular Formula of C₁₅H₁₀O₅, a Molecular Weight of 270.237 g/mol (270.24 daltons), a Chemical Abstracts Service (CAS) Registry Number 446-72-0 and a Beilstein Registry Number 263823. It is commercially available from a number of sources, including DSM Nutritional Products, Inc. of Basel, Switzerland under Drug Master File (DMF) #19747 and further described in PIND #119322.

Genistein is available as a highly bioavailable nano-suspension from Humanetics Corporation of Edina, Minn. under the trademark BIO 300™. Manufacture and administration of this nano-suspension is described in US Patents Application Publications 2012/0164190 and 2012/0121654, both hereby incorporated by reference.

Indications

The tripartite treatment regimen of this invention can be beneficially employed with and is indicated in those instances where the combination of radiation therapy (RT) and androgen deprivation therapy (ADT) is recommended to treat prostate cancer. RT/ADT therapy is the current standard of care for those at high risk of relapse or metastasis of prostate cancer, and is rapidly becoming the standard of care for those at intermediate risk.

Administration

Administration Route

Gonadotropin-releasing hormonal oncologics suitable for use in androgen deprivation therapy for prostate cancer are generally prepared for and administered by intramuscular depot injection.

Genistein can be administered by virtually any of the commonly accepted practices for the administration of pharmaceutical preparations including specifically, but not exclusively, mucosal administration, oral consumption, ocular administration, subcutaneous injection, transdermal administration, intravascular administration, intramuscular administration, etc. Oral administration as a nanosuspension is generally preferred.

Mucosal administration of genistein includes such routes as buccal, endotracheal, nasal, pharyngeal, rectal, sublingual, vaginal, etc. For administration through the buccal/sublingual/pharyngeal/endotracheal mucosal, genistein may be formulated as an emulsion, gum, lozenge, spray, tablet or an inclusion complex such as cyclodextrin inclusion complexes. Nasal administration is conveniently conducted through the use of a sniffing powder or nasal spray. For rectal and vaginal administration, genistein may be formulated as a cream, douche, enema or suppository.

Oral consumption of genistein may be effected by incorporating the genistein into a food or drink, or formulating the genistein into a chewable or swallowable tablet or capsule. The genistein is preferably orally administered as a nanosuspension in accordance with US Patent Application Publications 2012/0164190 and 2012/0121654, both hereby incorporated by reference.

Genistein is virtually insoluble in water, thereby limiting its bioavailability when administered orally. Genistein provided as a nanosuspension in accordance with US Patent Application Publications 2012/0164190 and 2012/0121654 has significantly improved oral bioavailability. This allows dosing without medical supervision, which enables pre-dosing at home prior to known and planned instances of radiation therapy. To further improve oral bioavailability, genistein can also be incorporated as sub-micron size particles in an orally ingestible formulation. Generally, a dose of ˜0.5 to 1 g per day of genistein provided as a nanosuspension should be effective for achieving the desired mitigating protective effect.

Ocular administration may be effected by incorporating genistein into a solution or suspension adapted for ocular application such as drops or sprays.

Subcutaneous, intravascular and intramuscular administration involves incorporating the genistein into a pharmaceutically acceptable and injectable carrier.

For transdermal administration, the genistein may be conveniently incorporated into a lipophilic carrier and formulated as a topical cream or adhesive patch.

Dosing Regimen

The radiation therapy and androgen deprivation therapy can be provided in accordance with standard industry practices for the treatment of prostate cancer. An exemplary dosing regimen for treatment of a high risk prostate cancer patient using a combination of radiation and androgen deprivation therapy is about 1.8-2.0 Gy/day 5 days per week for 7-8 weeks, accompanied by the neoadjuvant administration of LUPRON DEPOT@ 7.5 mg for 1-month, 22.5 mg for 3-month, 30 mg for 4-month, or 45 mg for 6-months, for 2-3 years. Administration of a gonadotropin-releasing hormonal oncologic is generally continued for a period of time after commencement of radiation therapy, with duration dictated in large part by the risk level for relapse or metastasis, and the extent to which the patient suffers from the adverse side-effects associated with prolonged androgen deprivation therapy. Generally, androgen deprivation therapy is continued for at least four weeks, and for high risk patients up to as long as 3 years after commencement of radiation therapy.

The desired synergistic effect of the tripartite combination therapy can generally be achieved by administration of at least ˜0.5 to 1 gram of genistein per day, preferably at least ˜1.2 grams of genistein per day and most preferably at least ˜1.5 grams of genistein per day, taken as a single dose or multiple doses each day. Lower amounts may also be therapeutic.

Genistein administration may commence prior to or shortly after commencement of radiation therapy. Genistein is preferably administered as a neoadjuvant, preferably administered at least one day and most preferably between three to seven days prior to commencement of fractionated radiation therapy, continued as a concomitant adjuvant throughout the radiation therapy, and preferably extended at least four weeks beyond conclusion of radiation therapy. Administration of genistein may be discontinued prior to conclusion of androgen deprivation therapy, but is preferably continued for at least so long as the androgen deprivation therapy is continued.

Genistein, as an adjuvant to the combination therapy of fractionated radiation therapy and androgen deprivation therapy for the treatment of prostate cancer, provides a synergistic therapeutic effect that not only improves treatment outcomes but reduces metastasis. This synergistic effect permits the dosing regimen of the gonadotropin-releasing hormonal oncologic (i.e., unit dose, frequency of administration and/or duration of administration) to be reduced relative to conventional dosing regimens for the oncologic, with minimal if any loss in treatment efficacy. Such a reduction in the dosing regimen of the gonadotropin-releasing hormonal oncologic provides a corresponding elimination or alleviation of the various adverse side effects associated with androgen deprivation therapy, particularly those associated with prolonged androgen deprivation therapy. Excellent efficacy can be maintained, with a concomitant elimination or alleviation of adverse side effects, with up to an 80% reduction in the dosing regimen of gonadotropin-releasing hormonal oncologic. Generally, a reduction of about 20% to 50% can provide excellent elimination or alleviation of adverse side effects with little or no diminishment in efficacy, while a reduction of greater than 50% up to as much as 80% can provide exceptional elimination or alleviation of adverse side effects with a modest reduction in efficacy.

Genistein may be administered at a reduced dosing regimen at some point after completion of radiation therapy, with the reduction preferably occurring only for the latter half of the post exposure time period during which genistein is administered. The reduction may be in the form of a reduced dosage (e.g., reduced to less than 60% the amount administered during the therapeutic stage) and/or a reduced frequency (e.g., ½ or ¼ the frequency during the therapeutic stage). When employed, this period of reduced administration should last for at least one month, preferably at least three months and most preferably at least six months. Shorter durations tend to diminish the benefit obtained by administration of the reduced dosage, while administration of some reduced dosage of genistein can perpetually benefit the patient.

1. A treatment regimen for a patient diagnosed with prostate cancer, comprising a combination therapy including at least (-) fractionated radiation therapy, (-) administration of a therapeutic amount of genistein, and (-) administration of a therapeutic amount of a gonadotropin-releasing hormonal oncologic.

2. The treatment regimen of claim 1 wherein the gonadotropin-releasing hormonal oncologic has a conventional androgen deprivation therapy dosing regimen, and the gonadotropin-releasing hormonal oncologic is administered at a reduced dosing regimen relative to the conventional androgen deprivation therapy dosing regimen.

3. The treatment regimen of claim 1 wherein the administration of genistein and the administration of a gonadotropin-releasing hormonal oncologic commence prior to the commencement of fractionated radiation therapy.

4. The treatment regimen of claim 1 wherein the administration of genistein and the administration of a gonadotropin-releasing hormonal oncologic commence at least 1 day prior to the commencement of fractionated radiation therapy.

5. (canceled)

6. (canceled)

7. The treatment regimen of claim 1 wherein the administration of genistein and the administration of a gonadotropin-releasing hormonal oncologic continue for at least 4 weeks after commencement of fractionated radiation therapy.

8. The treatment regimen of claim 2 wherein the administration of genistein and the administration of a gonadotropin-releasing hormonal oncologic continue at least until the conclusion of fractionated radiation therapy.

9. The treatment regimen of claim 1 wherein the administration of genistein and the administration of a gonadotropin-releasing hormonal oncologic continue for at least 1 year after commencement of fractionated radiation therapy.

10. (canceled)

11. The treatment regimen of claim 4 wherein genistein is administered at a unit dose during radiation therapy, and at a diminished unit dose for at least the latter half of the administration period occurring after completion of radiation therapy.

12. (canceled)

13. (canceled)

14. The treatment regimen of claim 1 wherein the administration of genistein continues for at least 4 weeks after commencement of fractionated radiation therapy and the administration of a gonadotropin-releasing hormonal oncologic continues for at least 4 months after commencement of fractionated radiation therapy.

15. The treatment regimen of claim 2 wherein the administration of genistein continues for at least 4 weeks after commencement of fractionated radiation therapy and the administration of a gonadotropin-releasing hormonal oncologic continues for at least 1 year after commencement of fractionated radiation therapy.

16. (canceled)

17. The treatment regimen of claim 1 wherein administration of a therapeutic amount of genistein comprises administration of at least 1 gram per day of genistein.

18. (canceled)

19. The treatment regimen of claim 3 wherein administration of a therapeutic amount of genistein comprises administration of at least 1.5 gram per day of genistein.

20. The treatment regimen of claim 1 wherein the genistein is administered orally in the form of a nanosuspension.

21. The treatment regimen of claim 1 wherein the gonadotropin-releasing hormonal oncologic is a gonadotropin-releasing hormone receptor agonist.

22. The treatment regimen of claim 1 wherein the gonadotropin-releasing hormone receptor agonist is a leuprolide.

23. The treatment regimen of claim 2 wherein the gonadotropin-releasing hormonal oncologic is administered at a reduced dosing regimen that is less than 80% of the conventional androgen deprivation therapy dosing regimen.

24. (canceled)

25. The treatment regimen of claim 2 wherein the gonadotropin-releasing hormonal oncologic is administered at a reduced dosing regimen that is less than 50% of the conventional androgen deprivation therapy dosing regimen.

26. (canceled)

27. The treatment regimen of claim 3 wherein the reduced dosing regimen is achieved by a reduction in unit dose.

28. The treatment regimen of claim 4 wherein the reduced dosing regimen is achieved by a decrease in frequency of administration.

29. (canceled)

30. The treatment regimen of claim 23 wherein the reduced dosing regimen is achieved by both a reduction in unit dose and a decrease in frequency of administration.
